(19) United States
(12) Patent Application Publication
(10) Pub. No.: US 2005/0175641 A1
(43) Pub. Date: Aug. 11, 2005

(54) Percutaneous and perungual delivery system

(75) Inventors: Peter Paul Deo, Oakleigh (AU); Albert Zorko Abram, Wantirna (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Connetics Australia Pty Ltd, Rowville (AU)

(21) Appl. No.: 10/941,054

(22) Filed: Sep. 10, 2004

(30) Foreign Application Priority Data
Jul. 31, 2002 (AU) 2002950506

(51) Int. Cl. A61K 7/04; A61K 9/14

(52) U.S. Cl. 424/400; 424/486; 424/488

(57) ABSTRACT

The invention relates to a substantially homogeneous liquid composition capable of percutaneous delivery of one or more physiologically active agents, the composition including at least one physiologically active agent, a volatile solvent, and a rate modulating carrier comprising a hydrophilic polymer and a hydrophobic polymer the combination of the hydrophilic and hydrophobic polymers being elected to enable modulation of the rate of physiologically active agent. The invention also relates to method of delivering an effective amount of an active agent and method of treatment of a patient using the composition of the invention.

### FIELD OF THE INVENTION

The present invention is concerned with a system suitable for the percutaneous and/or perungual delivery, particularly transdermal and/or transungual delivery of an active agent. The invention also relates to a method of percutaneous and perungual delivery of active agents and to therapeutic or prophylactic methods of treatment of a subject by percutaneous or perungual delivery of an active agent. The invention particularly relates to delivery of anaesthetics, anti infectives such as anti fungals, anti bacterials, anti virals, and anti acne agents. The delivery of anti acne agents such as Tretinoin (vitamin A acid) and anti-onychomycoses agents is particularly contemplated.

### BACKGROUND TO THE INVENTION

The term “active agent” as used herein is intended to denote substances that have a physiological effect, for example, a drug. The term “homogenous” as used herein is intended to mean uniform throughout. The term “film forming” as used herein is intended to mean a substance capable of forming a thin layer on the surface to which it is applied when exposed to ambient conditions. The term “liquid” as used herein is intended to mean a substance which is flowable. The term “percutaneous” as used herein is intended to mean any route of administering an active agent onto, into or through the skin of a subject so as to achieve one or more of a topical, local or systemic physiological effect. The term “perungual” as used herein is intended to mean any route of administering an active agent onto, into or through the keratinized ungual layer of the nail of a subject so as to achieve a topical or local physiological effect.

The use of the skin as a route for delivery of drugs is of relatively recent origin. One form of delivery system is that based on the use of an adhesive transdermal patch. These transdermal patches provide an alternative non-invasive parenteral route for the delivery of drugs which may or may not be suitable for oral administration. An example of an early form of a transdermal patch is described in U.S. Pat. No. 3,598,122 where the patch is in the form of a bandage.

Conventional routes of drug administration suffer several disadvantages when compared to the percutaneous route of drug administration. The percutaneous route of delivery may allow for the controlled release of an active agent into the systemic circulation. Many drugs are poorly absorbed by traditional routes of delivery and it has been found that the percutaneous route provides an effective method of achieving improved bioavailability for those active agents.

Topical creams for delivery of active agents being an alternative means of delivery of drugs for treatment of certain skin diseases are known. One such disclosure is that of U.S. Pat. No. 4,935,241 in the name of SHIONOGI & CO LTD. This patent describes a pharmaceutical formulation for localised treatment of tinea pedis which comprises a topical cream including an active agent and an ethyl acrylate-methyl methacrylate copolymer.

Another disclosure in this field is U.S. Pat. No. 6,211,250 assigned to Soltec Research Pty. Ltd. This patent describes a formulation for delivery of active agents percutaneously, specifically excluding the use of ethyl cellulose due to its lack of viscosity and inherent working difficulties.

The problem with known perungual methods of treatment, for example, of onychomycoses is that of ensuring that the antifungal agent penetrates into and beneath the nail to reach the site of infection. U.S. Pat. No. 6,143,793 in the name of Novartis AG is directed to the use of hydrophilic penetration agents in dermatological compositions for the treatment of onychomycoses, and is directed to improving the penetration of active agents in particularly difficult applications such as onychomycoses. The content of U.S. Pat. No. 6,143,793 is hereby incorporated by cross reference.

An objective of the present invention is to provide a system for the percutaneous and/or perungual delivery of one or more active agents which has any one or more of the following advantages. The system is desirably non-occlusive, rate variable and effective in delivering an active agent to have a systemic, topical or local effect upon a subject, particularly in the treatment of acne, or onychomycoses.

### SUMMARY OF THE INVENTION

Accordingly, the present invention provides, in one aspect, a substantially homogenous liquid composition capable of percutaneous and/or prungual delivery of one or more physiologically active agents, the composition comprising a volatile solvent, at least one physiologically active agent, and a rate modulating carrier comprising, at least two polymers, one of which is water soluble, and one of which is selected to enable modulation of the rate of delivery of said active agent. In certain preferred aspects, one of the at least two polymers of the rate modulating carrier is a modulation polymer.

The substantially homogenous liquid composition of the invention wherein said rate modulating carrier preferably comprises:
i) a hydrophilic polymer selected from the group consisting of polyvinyl alcohol, hydroxypropylmethyl cellulose, hydroxypropyl cellulose, polyvinyl pyrollidone, carbomer, PVM/MA decadiene cross polymer and hydroxypropylguar and copolymers thereof, said hydrophilic polymer being present in an amount of from 0.001 to 50% w/w of the total liquid composition; and
ii) a hydrophobic polymer being ethyl cellulose and being present in an amount of from 0.001 to 50% w/w of the total liquid composition,
the combination of hydrophilic and hydrophobic polymers being selected to enable modulation of the rate of delivery of said physiologically active ingredient.

The hydrophilic polymer is preferably hydroxypropyl cellulose.

The physiologically active agent is preferably an anaesthetic, anti infective or anti-acne agent, more preferably an anti acne agent, or an anti-onychomycoses agent and more preferably still one or more retinoids such as tretinoin, or one or more allylamines such as terbinafine or naftifine.

An advantage of the present invention is that the composition of the invention can be dispensed onto, and smoothed onto the skin or nail of a subject to form a thin film on the skin or nail surface, this film providing for the percutaneous or perungual delivery of the one or more active agents contained in the composition. Unlike conventional transdermal patches, the transdermal system of the present invention does not require the use of an adhesive layer. Moreover, it is robust (resistant to accidental removal), waterproof and has good substantivity on the skin. This makes it well suited to perungual applications as well as where paints are conventionally used. It has additionally been found that the formulations according to the invention can be varied by altering the nature of the modulating carrier to alter the rate of release of the active agent into the skin or nail of the patient. In particular it is found that the use of the modulating carrier enables the formation of a reservoir of active agent on the skin or in the nail of the patient which can be absorbed by the skin or nail at a varying rate depending on the other components of the formulation.

In percutaneous applications, although it is preferred that the skin surface to which the composition is to be applied be non-hairy, the presence of hair does not create as significant a problem as is the case with adhesive patches. Similarly the presence of wrinkles, creases and folds in the skin is not an impediment to the application of the composition of the invention to a particular area of the body, although it is preferable to avoid areas that have significant creasing or folds. Moreover the film that is formed is unobtrusive to the subject in that the subject is not significantly aware of its presence on the skin.

The composition is preferably such that when applied to the skin or nail, the volatile solvent will evaporate which may leave a two-phase film. The formed film may include a continuous phase and a dispersed phase. The hydrophilic polymer may form the continuous phase and the hydrophobic polymer may form the disperse phase in the formed film, or vice versa.

Where the composition of the invention is used to form a two-phase film, the active agent may be contained in the continuous phase of the film or in the dispersed phase, or in both phases. It is thought that the inclusion of the active agent in the continuous phase of a formed film has the effect of increasing the release rate of the active whereas including the active in the dispersed phase slows down its rate of release.

The composition of the invention advantageously also comprises a thickening agent. Preferably said thickening agent is soluble in both water and alcohol. More preferably, the thickening agent is a polymer, preferably a hydrophilic polymer. Preferably the hydrophobic polymer is a modulating polymer, very preferably ethyl cellulose.

A preferred ratio of modulating polymer:active is 1-10,000:10,000-1. The ratio will vary according to the potency of the active agent, i.e. how much active agent on a mass basis is required to achieve the physiological effect desired.

A preferred ratio of hydrophobic polymer:hydrophilic polymer is 1-100:100-1. A more preferred ratio of hydrophobic polymer:hydrophilic polymer is 1-10:10-1.

In certain other aspects, the present invention provides compositions, systems and methods for modulating the rate or flux of the delivery of an active agent. In certain aspects, the rate or flux such as the transdermal rate or flux, is predetermined and designed for a particular active agent. Advantageously, by selecting a particular modulating polymer, a specific predetermined rate or flux of percutaneous and/or perungual delivery of an active agent can be built into the system.

The compositions of the invention may include one or more skin or nail absorption/penetration enhancers which enhance the absorption and/or penetration of the active agent. The absorption/penetration enhancers may be present in an amount of about 0.1 to 40% w/w of the composition. The absorption/penetration enhancer may be any suitable enhancer known in the art. The rate of penetration of the active agent may also be varied by adjusting the rate of release of the penetration enhancer from the polymer.

The composition of the invention may be in the form of a solution or a dispersion. The composition may also be in the form of a gel.

Where the composition is in the form of a dispersion, the dispersed phase may be in the form of nanoparticles, microparticles, microcapsules, microspheres, microsponges or liposomes which may contain (in whole or in part) and/or be coated with the active agent. The active agent may be a combination of agents. Where the dispersed phase is in the form of nanoparticles, microparticles, microcapsules, microspheres or liposomes, the continuous phase may include a hydrophobic polymer or a hydrophilic polymer. The active agent may be dispersed or dissolved in the composition of the invention and may be present in the composition in a physiologically effective amount. The concentration of active agent used in the composition of the invention may be approximately equivalent to that normally utilised for that particular agent in conventional formulations, particularly that used in conventional transdermal patch delivery systems or paints. The amount of an active agent to be incorporated in the composition varies depending on the particular active agent, the desired therapeutic effect, and the time span for which the system is to provide therapy. For most active agents, the passage of the drugs through the skin or nail will be the rate-limiting step in delivery. Thus, the amount of active agent and the rate of release is typically selected so as to provide transdermal delivery characterised by a zero order time dependency for a prolonged period of time. The minimum amount of active agent in the system is selected based on the amount of active agent which passes through the skin or nail in the time span for which the device is to provide therapy.

Normally, the amount of active agent in the system can vary from about 0.01% w/w to about 50% w/w.

In the case of tretinoin, the active agent may be present in amounts of 0.01%-0.5% w/w.

The compositions may include other excipients such as antioxidants, stabilisers, plasticisers and waterproofing agents. Such excipients include but are not limited to butylated hydroxy toluene and triethyl citrate.

In another aspect, the present invention provides a method for the prophylactic or therapeutic treatment of a patient comprising percutanenusly or perungually delivering an effective amount of an active agent by application of a composition in accordance with the present invention to the skin or nail of the subject. The subject may be human or an animal. The subject may be suffering a systemic or a localised medical condition. In a preferred embodiment of this aspect of the invention, the subject may be suffering from acne or onychomycoses.

In still another aspect of the invention, there is provided the use of a composition according to the invention for the prophylactic or therapeutic treatment of a patient having a medical condition treatable via the skin. The patient may be suffering a systemic or local medical condition. In a preferred embodiment the patient may be suffering from acne or onychomycoses.

The composition according to the invention may have an anti fungal, anti bacterial, anti viral, anti-acne or keratolytic activity. The rate of delivery of said physiologically active agent is adjustable by varying the ratio of said modulating polymer with respect to the active agent. The rate of delivery of said physiologically active agent is adjustable by varying the ratio of said hydrophobic polymer with respect to the hydrophilic polymer.

The system can be used with transdermal penetration enhancers to modify the transdermal flux rate of active molecules. It can also be used with or without penetration enhancers to effectively retain active substances on the top layers of skin or nail or to provide a sustained rate of release of active into the skin or nail.

### DETAILED DESCRIPTION OF THE INVENTION

The volatile solvent used in the compositions of the invention may be one or more pharmaceutically or veterinarally acceptable solvents. The solvent may be present in an amount of at least 50% w/w. Examples of suitable volatile solvents include skin safe solvents such as a lower alkanol (e.g. ethanol, isopropanol and the like), acetone, water, or mixtures thereof.

The hydrophilic polymer or thickening agent be selected from any pharmaceutically or veterinarally acceptable polymer. Examples of suitable hydrophilic polymer or thickening agent include substitutes alkylcellulose such as hydroxyalkyl cellulose. Other hydrophilic polymers may be used, e.g. Polyvinyl alcohol, polyvinylpyrrolidone, carbomer, PVM/MA decadiene cross polymer, hydroxypropylguar etc. The hydrophilic polymer or thickening agent is preferably hydroxypropyl cellulose.

The hydrophobic polymer is preferably ethyl cellulose.

The overall polymer content of the composition of the invention may be up to 50% W/W.

The hydrophilic polymer, or thickening agent may present in an amount of 0.001 to 50% w/w of the total liquid composition, and preferably present in an amount of about 0.05 to 30% w/w of the composition of the invention. More preferably, the hydrophilic polymer is present in an amount of 0.05 to 10% w/w of the composition, most preferably 0.1 to 5.0% w/w of the composition.

The hydrophobic polymer may be present in an amount up to about 50% w/w. The hydrophobic polymer may be present in an amount of about 0.001 to 30% of the composition of the invention. Preferably, the hydrophobic polymer is present in an amount of 0.05 to 20% of the composition of the invention, more preferably 0.05 to 10% of the composition, more preferably 0.05 to 6.0%.

In determining the amount of each polymer, and the ratio of hydrophobic to hydrophilic polymer, there are some general guidelines. The final levels must be confirmed by empirical tests of skin penetration, but as a general rule the higher the overall level of the hydrophobic polymer, and the higher the ratio of hydrophobic to hydrophilic polymer, the slower the penetration, and the more active agent that will accumulate in the epidermis rather than be carried through into the dermis. However, non-polar active agents will dissolve more readily in the hydrophobic polymer and may give different results from more polar active agents.

Based upon the physical characteristics of the active (log P, hydrophilicity, hydrophobicity, and the like) the hydrophobic and hydrophilic polymers would be adjusted to deliver a specific rate or flux for a specific active. It may be desirable to deliver more or less of a specific active to a specific spot based upon toxicity, dosage etc. Given the various parameters and physical characteristics of the compositions, it is envisaged that a specific composition could be prepared for a specific application for the specific job.

The active agent may be any suitable pharmaceutically effective compound, but preferably an anaesthetic, an anti infective such as an antifungal, antibacterial or antiviral, more preferably an allylamine such as terbinafine or naftifine or an antic acne agent such as a retinoid and more preferably tretinoin. The active agent may alternatively be a drug that is normally delivered by oral, parenteral, percutaneous, perungual or rectal route. The active agent may be a prodrug.

Other examples of active drugs that can be administered by the novel transdermal drug delivery system of this invention include, but are not limited to:

Cardioactive medications, for example, organic nitrates such as nitroglycerine, isosorbide dinitrate, and isosorbide mononitrates; quinidine sulfate; procainamide; thiazides such as bendroflumethiazide, chlorothiazide, and hydrochlorothyazide; nifedipine; nicardipine; adrenergic blocking agents, such as timolol and propranolol; verapamil; diltiazem; captopril; clonidine and prazosin.

Androgenic steroids, such as testosterone, methyltestosterone and fluoxymesterone.

Estrogens, such as conjugated estrogens, esterified estrogens, estropipate, 17beta estradiol, 17beta-estradiol valerate, equilin, mestranol, estrone, estriol, 17beta-ethinyl estradiol, and diethylstilboestrol. Progestational agents, such as progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17alpha hydroxyprogesterone, dydrogesterone, dimethisterone, ethinylestrenol, norgestrel, demegestone, promegestone, and megestrol acetate.

Drugs having an action on the central nervous system, for example sedatives, hypnotics, antianxiety agents, analgesics and anaesthetics, such as chloral, buprenorphine, naloxone, haloperidol, fluphenazine, pentobarbital, phenobarbital, secobarbital, codeine, lidocaine, tetracaine, dyclonine, dibucaine, methocaine, cocaine, procaine, mepivacaine, bupivacaine, etidocaine, prilocaine, benzocaine, fentanyl, and nicotine.

Nutritional agents, such as vitamins, essential amino adds and essential fats.

Anti-flammatory agents, such as hydrocortisone, cortisone, dexamethasone, fluocinolone, triamcinolone, medrysone, prednisolone, flurandrenolide, prednisone, halcinonide, methylprednisolone, flurandrenolide, prednisone, halcinonide, methylprednisolone, fludrocortisone, corticosterone, paramethasone, betamethasone, ibuprofen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like.

Antihistamines, such as diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, and chlorpheniramine.

Respiratory agents, such as theophilline and beta2-adrenergic agonists such as albuterol, terbutaline, metaproterenol, ritodrine, carbuterol, fenoterol, quinterenol, rimiterol, solmefamol, soterenol, and tetroquinol.

Sympathomimetics, such as dopamine, norepinephrine, phenylpropanolamine, phenylephrine, pseudoephedrine, amphetamine, propylhexedrine and epinephrine. Miotics, such as pilocarpine, and the like. 12 Cholinergic agonists, such as choline, acetylcholine, methacholine, carbachol, bethanechol, pilocarpine, muscarine, and arecoline.

Antimuscarinic or muscarinic cholinergic blocking agents such as atropine, scopolamine, homatropine, methscopolamine, homatropine methylbromide, methantheline, cyclopentolate, tropicamide, propantheline, anisotropine, dicyclomine, and eucatropine. Mydriatics, such as atropine, cyclopentolate, homatropine, scopolamine, tropicamide, eucatropine and hydroxyamphetamine.

Psychic energizers such as 3-(2-aminopropyl)indole, 3(2-aminobutyl)indole, and the like.

Anti-infectives, such as antivirals, eg acyclovir, allylamines and in particular terbinafine hydrochloride and naftifine hydrochloride antibiotics, including penicillin, tetracycline, chloramphenicol, sulfacetamide, sulfamethazine, sulfadiazine, sulfamerazine, sulfamethizole and sulfisoxazole; antivirals, including idoxuridine; antibacterials, such as erythromycin and clarithromycin; and other anti-infectives including nitrofurazone and the like.

Dermatological agents, such as vitamins A and E.

Humoral agents, such as the prostaglandins, natural and synthetic, for example PGE1, PGF2alpha, and PGF2alpha, and the PGE1 analog misoprostol.

Antispasmodics, such as atropine, methantheline, papaverine, cinnamedrine, and methscopolamine.

Antidepressant drugs, such as isocarboxazid, phenelzine, tranylcypromine, imipramine, amitriptyline, trimipramine, doxepin, desipramine, nortriptyline, protriptyline, amoxapine, maprotiline, and trazodone.

Anti-diabetics, such as insulin, and anticancer drugs such as tamoxifen and methotrexate.

Anorectic drugs, such as dextroamphetamine, methamphetamine, phenylpropanolamine, fenfluramine, diethylpropion, mazindol, and phentermine. Anti-allergenics, such as antazoline, methapyrilene, chlorpheniramine, pyrilamine and pheniramine.

Tranquilizers, such as reserpine, chlorpromazine, and antianxiety benzodiazepines such as alprazolam, chlordiazepoxide, clorazeptate, halazepam, oxazepam, prazepam, clonazepam, flurazepam, triazolam, lorazepam and diazepam.

Antipsychotics, such as thiopropazate, chlorpromazine, triflupromazine, mesoridazine, piperacetazine, thioridazine, acetophenazine, fluphenazine, perphenazine, trifluoperazine, chlorprathixene, thiothixene, haloperidol, bromperidol, loxapine, and molindone.

Decongestants, such as phenylephrine, ephedrine, naphazoline, Antipyretics, such as aspirin, salicylamide, and the like.

Antimigraine agents, such as dihydroergotamine and pizotyline.

Drugs for treating nausea and vomiting, such as chlorpromazine, perphenazine, prochlorperazine, promethazine, scopolamine, hyacine hydrobromide, triethylperazine, triflupromazine, and trimeprazine.

Anti-malarials, such as the 4-aminoquinolines, alpha-aminoquinolines, chloroquine, and pyrimethamine.

Anti-ulcerative agents, such as misoprostol, omeprazole, and enprostil.

Peptides and proteins, such as drugs for Parkinson's disease, spasticity, and acute muscle spasms, such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, insulin, erythropoietin and growth hormone.

Anti-estrogen or hormone agents, such as tamoxifen or human chorionic gonadotropin.

Nucleotides and nucleic acids (e.g. DNA).

The active agents can be present in the composition in different forms, depending on which form yields the optimum delivery characteristics. Thus, in the case of drugs, the drug can be in its free base or acid form, or in the form of salts, esters, or any other pharmacologically acceptable derivatives, or as components of molecular complexes.

### BRIEF DESCRIPTION OF THE FIGURES

FIG. 1 are representative histological pictures from the right and left ear of the hamsters in the three test groups from Example 19. Each group was subject to a separate treatment regime to deliver tretinoin to the ear.

FIG. 2 is a graph of the effect of drug concentrations on the amount of drug in the epidermis and in the receptor. The error bars represent the S.D. based on the average 3×6 data.

FIG. 3 is a graph of the effects of formulations on the amount of drug in the epidermis and in the receptor. The error bars represent the S.D. based on the average of 3×6 data.

FIG. 4 is a graph of the effects of formulations on the amount of drug in the epidermis and in the receptor. The error bars represent the S.D. based on the average of 3×6 data.

FIG. 5 is a plot of the cumulative amount of drug in the receptor fluid over time.

The following compositions were prepared in accordance with the invention by combining the following components in a stirred vessel at ambient temperature. It will be appreciated that the invention is not limited to the specific embodiments set out hereinbelow which are merely representative of the scope of the invention.

Examples 1-3 demonstrate the same basic formula (0.1% Tretinoin) with 3 different antioxidant types (namely BHA, BHT and tocopherol)

Examples 4-7 demonstrate various Hydrophobic polymer (EC) to active ratios (from 1:1 through to 40:1) for the same basic formula (0.1% Tretinoin) used in examples 1-3

Example 8 demonstrates a Hydrophobic polymer (EC) to active ratio of 80:1 (4% EC with 0.05% Tretinoin)

Example 9 demonstrates an anti-viral suspension composition

Examples 10-11 demonstrates some anaesthetic compositions (5% Lidocaine and 1% Lidocaine respectively)

Example 12 demonstrates a Hydrophobic polymer (EC) to active ratio of 160:1 (4% EC with 0.025% Tretinoin)

Example 13 demonstrates an antifungal composition

Examples 14-18 demonstrates the use of a fixed amount of hydrophobic polymer (EC) with various types and concentrations of hydrophilic polymers for antibacterial compositions

Example 19 demonstrates the use of a combination of active agents.

### EXAMPLE 1

| Item No. | Ingredient | % w/w |
|---|---|---|
| 1. | Tretinoin | 0.10 |
| 2. | Triethyl Citrate | 0.50 |
| 3. | Butylated Hydroxyanisole | 0.10 |
| 4. | Ethyl Cellulose | 1.00 |
| 5. | Hydroxypropyl Cellulose | 2.00 |
| 6. | Ethanol 95% | To 100 |
| | Total | 100.00 |

### EXAMPLE 2

| Item No. | Ingredient | % w/w |
|---|---|---|
| 1. | Tretinoin | 0.10 |
| 2. | Triethyl Citrate | 0.50 |
| 3. | Butylated Hydroxytoluene | 0.10 |
| 4. | Ethyl Cellulose | 1.00 |
| 5. | Hydroxypropyl Cellulose | 2.00 |
| 6. | Ethanol 95% | To 100 |
| | Total | 100.00 |

### EXAMPLE 3

| Item No. | Ingredient | % w/w |
|---|---|---|
| 1. | Tretinoin | 0.10 |
| 2. | Triethyl Citrate | 0.50 |
| 3. | Tocopherol | 0.10 |
| 4. | Ethyl Cellulose | 1.00 |
| 5. | Hydroxypropyl Cellulose | 2.00 |
| 6. | Ethanol 95% | To 100 |
| | Total | 100.00 |

### EXAMPLE 4

| Item No. | Ingredient | % w/w |
|---|---|---|
| 1. | Tretinoin | 0.10 |
| 2. | Triethyl Citrate | 0.50 |
| 3. | Butylated Hydroxytoluene | 0.10 |
| 4. | Ethyl Cellulose | 0.05 |
| 5. | Hydroxypropyl Cellulose | 2.00 |
| 6. | Ethanol 95% | To 100 |
| | Total | 100.00 |

### EXAMPLE 5

| Item No. | Ingredient | % w/w |
|---|---|---|
| 1. | Tretinoin | 0.10 |
| 2. | Triethyl Citrate | 0.50 |
| 3. | Butylated Hydroxytoluene | 0.10 |
| 4. | Ethyl Cellulose | 0.25 |
| 5. | Hydroxypropyl Cellulose | 2.00 |
| 6. | Ethanol 95% | To 100 |
| | Total | 100.00 |

### EXAMPLE 6

| Item No. | Ingredient | % w/w |
|---|---|---|
| 1. | Tretinoin | 0.10 |
| 2. | Triethyl Citrate | 0.50 |
| 3. | Butylated Hydroxytoluene | 0.10 |
| 4. | Ethyl Cellulose | 2.00 |
| 5. | Hydroxypropyl Cellulose | 2.00 |
| 6. | Ethanol 95% | To 100 |
| | Total | 100.00 |

### EXAMPLE 7

| Item No. | Ingredient | % w/w |
|---|---|---|
| 1. | Tretinoin | 0.10 |
| 2. | Triethyl Citrate | 0.50 |
| 3. | Butylated Hydroxytoluene | 0.10 |
| 4. | Ethyl Cellulose | 4.00 |
| 5. | Hydroxypropyl Cellulose | 2.00 |
| 6. | Ethanol 95% | To 100 |
| | Total | 100.00 |

### EXAMPLE 8

| Item No | Ingredient | % w/w |
|---|---|---|
| 1. | Ethanol 100% | 86.85 |
| 2. | Tretinoin | 0.05 |
| 3. | Butylated Hydroxy | 0.10 |
| | Toluene | |
| 4. | Triethyl Citrate | 0.50 |
| 5. | Ethyl Cellulose | 4.00 |
| 6. | Water | 6.50 |
| 7. | Hydroxypropylcellulose | 2.00 |
| | | 100.00 |

### EXAMPLE 9

| Item No | Ingredient | % w/w |
|---|---|---|
| 1. | Acyclovir | 5.0 |
| 2. | Ethanol | 89.5 |
| 3. | Carbopol 940 | 0.5 |
| 4. | Ethyl Cellulose | 1.0 |
| 5. | Hydroxypropylcellulose | 2.5 |
| 6. | Ethonium 25 | 1.5 |

### EXAMPLE10

| Item No | Ingredient | % w/w |
|---|---|---|
| 1. | Ethanol 95% | 87.90 |
| 2. | Glycerol | 5.00 |
| 3. | Lidocaine | 5.00 |
| 4. | Hydroxypropylcellulose | 2.00 |
| 5. | Ethyl cellulose | 0.10 |

### EXAMPLE 11

| Item No | Ingredient | % w/w |
|---|---|---|
| 1. | Isopropanol | 96.90 |
| 2. | Lidocaine | 1.00 |
| 3. | Hydroxypropylcellulose | 2.00 |
| 4. | Ethyl cellulose | 0.10 |

### EXAMPLE 12

| Item No. | Ingredient | % w/w |
|---|---|---|
| 1. | Tretinoin | 0.025 |
| 2. | Triethyl Citrate | 0.50 |
| 3. | Butylated Hydroxytoluene | 0.10 |
| 4. | Ethyl Cellulose | 4.00 |
| 5. | Hydroxypropyl Cellulose | 2.00 |
| 6. | Ethanol 95% | To 100 |
| | Total | 100.00 |

### EXAMPLE 13

| Item No. | Ingredient | % w/w |
|---|---|---|
| 1. | Ethanol 100% | 85.03 |
| 2. | Water | 6.97 |
| 3. | Ethyl Cellulose | 1.00 |
| 4. | Ketoconazole | 2.00 |
| 5. | PVP K30 | 5.00 |
| | Total | 100.00 |

### EXAMPLE 14

| Item No. | Ingredient | % w/w |
|---|---|---|
| 1. | Ethanol 100% | 89.65 |
| 2. | Water | 7.35 |
| 3. | Ethyl Cellulose | 1.00 |
| 4. | Mupirocin | 1.00 |
| 5. | Jaguar HP-120 | 1.00 |
| | Total | 100.00 |

### EXAMPLE 15

| Item No. | Ingredient | % w/w |
|---|---|---|
| 1. | Ethanol 100% | 89.55 |
| 2. | Water | 7.35 |
| 3. | Ethyl Cellulose | 1.00 |
| 4. | Mupirocin | 2.00 |
| 5. | Klucel H | 0.10 |
| | Total | 100.00 |

### EXAMPLE 16

| Item No. | Ingredient | % w/w |
|---|---|---|
| 1. | Ethanol 100% | 88.72 |
| 2. | Water | 7.28 |
| 3. | Ethyl Cellulose | 1.00 |
| 4. | Mupirocin | 2.00 |
| 5. | Klucel H | 1.00 |
| | Total | 100.00 |

### EXAMPLE 17

| Item No. | Ingredient | % w/w |
|---|---|---|
| 1. | Ethanol 100% | 80.41 |
| 2. | Water | 6.59 |
| 3. | Ethyl Cellulose | 1.00 |
| 4. | Mupirocin | 2.00 |
| 5. | Klucel L | 10.00 |
| | Total | 100.00 |

### EXAMPLE 18

| Item No. | Ingredient | % w/w |
|---|---|---|
| 1. | Ethanol 100% | 71.16 |
| 2. | Water | 5.84 |
| 3. | Ethyl Cellulose | 1.00 |
| 4. | Mupirocin | 2.00 |
| 5. | Klucel L | 20.00 |
| | Total | 100.00 |

### EXAMPLE 19

| Item No. | Ingredient | % w/w |
|---|---|---|
| 1. | Ethanol 100AGF22 | 62.121 |
| 2. | Tretinoin | 0.025 |
| 3. | Triethyl Citrate | 0.500 |
| 4. | BHT | 0.100 |
| 5. | Ethyl Cellulose N-10 | 4.000 |
| 6. | Water Purified | 30.000 |
| 7. | Clindamycin phosphate | 1.254 |
| 8. | Klucel MF | 2.000 |
| | Total | 100.000 |

### EXAMPLE 20

### Penetration Studies

The objectives of the following study were to compare the therapeutic efficacy of a tretinoin, 0.1% formulation in accordance with example 7 with two commercially available products (Retin-A cream, 0.1% and Retin-A micro, 0.1%) on the size of sebaceous glands of hamster ear after topical application.

Materials and Methods

Test Animals were six male adult Syrian Hamsters weighing 150 g. The Hamsters were generally healthy and were acclimated for 5 days. During acclimation, the hamsters were observed at least once daily for clinical signs of abnormality. All Hamsters were housed individually and were identified by animal numbers on cage cards and by distinct marking on the animals' tails using indelible ink.

All hamsters were housed individually in micro-isolator cages in a pathogen free environment with a 12 hour light-dark cycle and free access to food and water ad libitum.

Study Design

| Group | | |
|---|---|---|
| No. | Test Article | Treatment duration |
| 1 | Retin-A cream, 0.1% | 15 μl, right ear, d0-14 |
| | tretinoin | |
| 2 | Retin-A micro, 0.1% | 15 μl, right ear, d0-14 |
| | tretinoin | |
| 3 | Formulation according to | 15 μl, right ear, d0-14 |
| | the invention 0.1% tretinoin | |
| | (hereinafter “Liquipatch” | |

Dose Preparation and Administration

On each morning during the experiment period, 15 μl of testing materials was applied to the ventral side of the right ear of hamsters under general gas anesthesia for 5 minutes, once daily for 14 days.

On day 14, all hampsters were anesthetized by the intraperitoneal injection of sodium pentobarbital (50 mg/kg). Both ears were removed by cutting at the base using surgical scissors. The dorsal ear skin was gently pulled away from the supporting cartilage, starting at the base and extending distally. The “stripped skin” was then placed in 10% phosphate-buffered formalin.

After fixation for 15 hours, the tissues were embedded in paraffin and sagittal sections were cut at 5-μm thickness. The sections were then stained with standard H&E staining and evaluated under microscope.

All animals were healthy during the experiment period. No observable side effects were observed.

The representative histological pictures from both ears (left and right) in each experimental group are showing in the following figures. All treatment groups demonstrated significant reduction on the size of sebaceous gland in the treated ear, but not in the control ear (left). The reduction of size among all treatments is comparable.

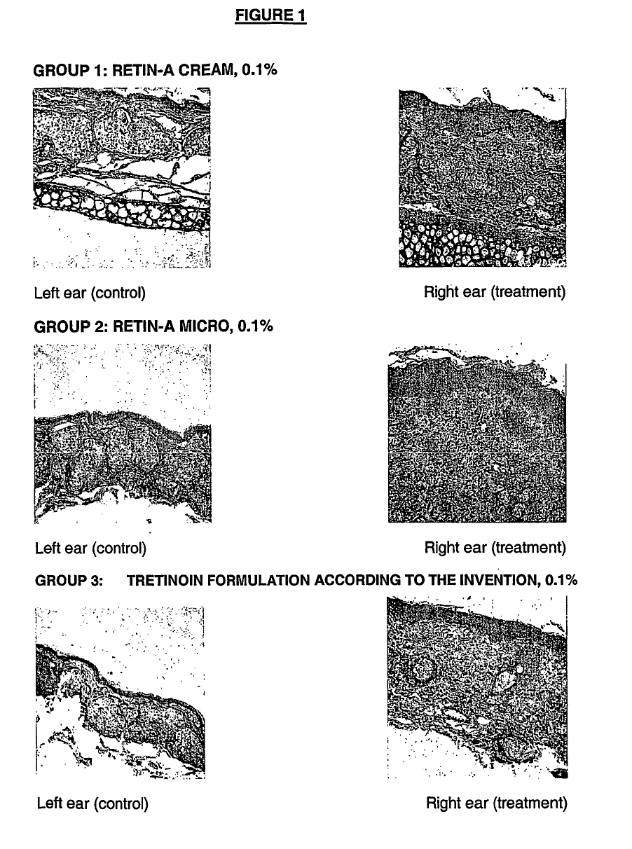

The thickness of epidermis among all treatment groups is slightly different. The thinnest one is after the treatment with a Tretinoin formulation according to the invention. The results are shown in FIG. 1.

Based on the histological observation from this pilot study, these results demonstrated that therapeutic efficacy of three treatment regimens are comparable in terms of effect on reducing sebaceous gland size in hamster ear after topical application. The formulabons according to the invention are therefore considered to be comparable in effect to the two commercially available products in histological tests.

### EXAMPLE 21

A further study was conducted to study tretinoin formulations according to the invention and their epidermal penetration in comparison with competing products.

| Formulation | | | |
|---|---|---|---|
| Number | Code | Formulation Name | Manufacturer |
| 1 | R-C-0.1 | 0.1% Retin-A Cream | Ortho |
| 2 | R-C-0.05 | 0.05% Retin-A Cream | Pharmaceutical |
| 3 | R-M-0.1 | 0.1% Retin-A Micro | Corp. |
| 4 | R-G-0.025 | 0.025% Retin-A Gel | |
| 5 | A-C-0.025 | 0.025% Avita Cream | Penederm, Inc. |
| 6 | A-G-0.025 | 0.025% Avita Gel | |
| 7 | L-G-0.1 | 0.1% Tretinoin gel | |
| | | *according to invention | |
| 8 | L-G-0.05 | 0.05% Tretinoin gel | |
| | | +according to invention | |
| 9 | L-G-0.025 | 0.025% Tretinoin gel | |
| | | !according to invention | |
| 10 | L-H-0.05 | 0.05% Tretinoin gel | |
| | | \hat{ }according to invention | |

Tretinoin gel formulations according to the invention
*= example 7
+= example 8
!= example 12
\hat{ }= example 8 without the hydrophobic polymer

Abbreviation for Texts:

R: Retin-A, A: Avita

L: Liquipatch, C: Cream G: Gel

Study Design:

| | |
|---|---|
| Membrane: | Heat separated human epidermis |
| Repeats: | n = 6 per formulation on 3 skins each |
| Donor phase: | 5-10 mg/cm² formulation spiked with 40-50 μCi |
| | radiolabeled tretinoin per 500 mg formulation |
| Receptor phase: | 4% BSA in PBS pH 7.4 |
| Sampling times: | 3, 6, 9, 12 & 24 hours |
| Balance: | Surface wash (1), swab (1), single tape strip and |
| | epidermal retention also determined. |
| Analysis: | HPLC verification of radiolabel |
| | Radioactive scintillation counting assay |

The studies revealed the following results wherein the term “LIQUIPATCH” or

“L-” refers to formulations according to the invention.

Results Average

| | | | | % washed | | | |
|---|---|---|---|---|---|---|---|
| | | | | swabbed | | % in | |
| | | Dosage | | and | % in | receptor | Total |
| No. | Code | Form | Drug Conc. | stripped | epidermis | fluid | recovered |
| 1 | R-C-0.1 | Cream | 0.100% | 69.1 | 1.9 | 0.08 | 71.1 |
| 2 | R-C-0.05 | Cream | 0.050% | 56.4 | 1.8 | 0.08 | 58.3 |
| 3 | R-M-0.1 | Micro | 0.100% | 89.3 | 1.9 | 0.09 | 91.2 |
| 4 | R-G-0.025 | Gel | 0.025% | 46.2 | 2.1 | 0.04 | 48.3 |
| 5 | A-C-0.025 | Cream | 0.025% | 54.3 | 0.9 | 0.05 | 55.2 |
| 6 | A-G-0.025 | Gel | 0.025% | 53.0 | 0.8 | 0.02 | 53.8 |
| 7 | L-G-0.1 | Gel | 0.100% | 53.1 | 1.2 | 0.02 | 54.3 |
| 8 | L-G-0.05 | Gel | 0.050% | 53.5 | 1.6 | 0.04 | 55.1 |
| 9 | L-G-0.025 | Gel | 0.025% | 53.4 | 1.5 | 0.04 | 54.9 |
| 10 | L-H-0.05 | Gel no | 0.050% | 51.0 | 2.5 | 0.06 | 53.5 |
| | | polymer | | | | | |

Table 1 Drug Distribution throughout Skin Diffusion System

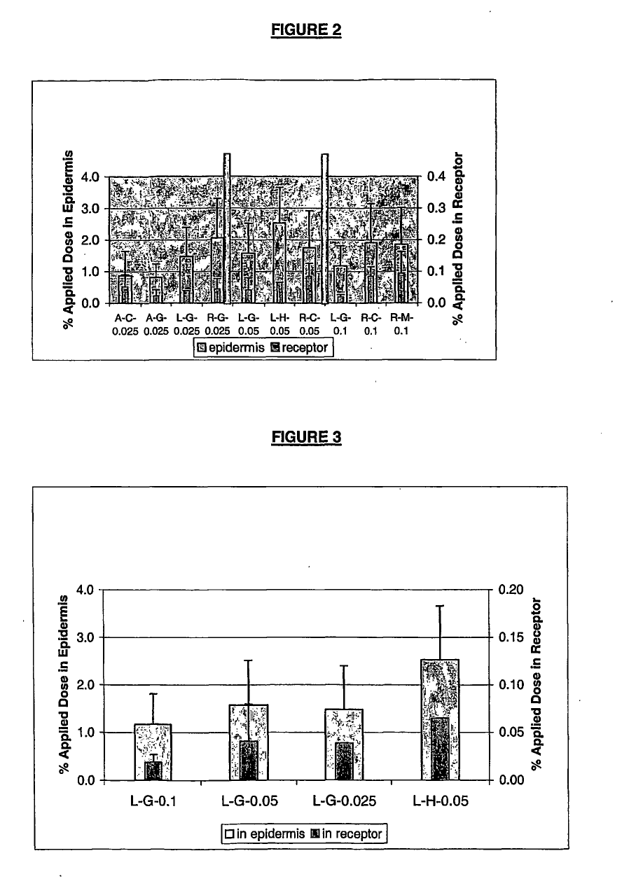

FIG. 2 shows that the drug distribution is independent of drug concentration. The Vehicle will impact on drug distribution.

FIG. 3 shows that the Liquipatch formulation has decreased the amount of tretinoin that has penetrated into the receptor fluid, but increased the amount seen in the epidermis. As the receptor fluid corresponds to the dermis, the lower level of tretinoin indicates less irritation, but the high level in the epidermis suggests good anti-acne efficacy. This shows that the combination of hydrophobic and hydrophilic polymers and other ingredients in the Liquipatch have given a product that should demonstrate low irritation, similar to the Avita product, but high anti-acne effect, similar to the Retin-A product.

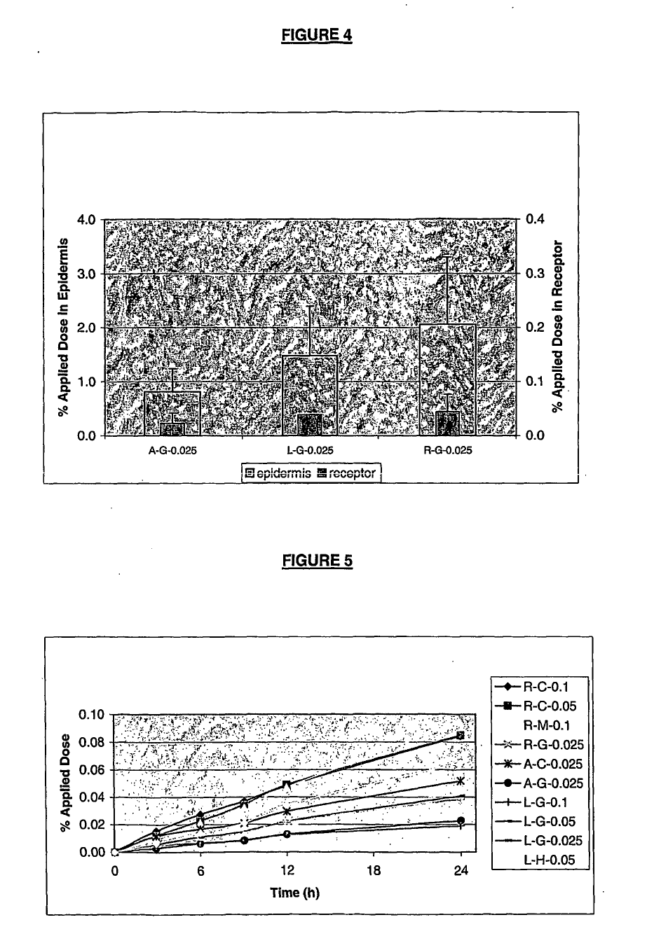

The results in FIG. 4 show that formulations according to the invention can deliver a comparable amount of active in the epidermis to Retin A, and more than that of Avita. The formulations according to the invention deliver an equivalent amount to Avita to the receptor, and a lower amount than does Retin A. it is generally known that Avita has lower irritation, however also has lower efficacy than Retin A. From a correlation of these results it could therefore be expected that the formulation of the invention has high efficacy (as does Retin A) while having low irritation similar to Avita.

FIG. 5 shows that comparing equivalent concentration of each of the three products, it can be seen that the penetration profile is independent of the drug concentration. The penetration appears to be more dependent upon the vehicle. The drug release rate from Liquipatch is slower than Retin-A and Avita

Conclusions

Liquipatch and Retin A delivered a similar amount to be retained in epidermis and more efficiently than Avita. However, Retin A allowed more drug passing through the epidermis into the receptor (at a higher flux), than Liquipatch and Avita.

Creams and gels delivered similar amount retained in epidermis, but creams yielded more drug passing the epidermis into the receptor chamber (at a higher flux) than gels.

Increasing concentrations in creams did not affect the percentage delivered, but in Liquipatch gels, 0.1% delivered less fraction in epidermis and receptor than the lower concentrations. Therefore, the drug concentration 0.05% in Liquipatch gel may be the most efficient in term of the amount penetrated to the amount applied.

Within Liquipatch data, the presence of hydrophobic polymer reduces the amount of drug penetrating the epidermis. The gel without hydrophobic polymer delivered more in epidermis and receptor chamber (at a higher flux). Moreover, the gel without hydrophobic polymer showed a similar release profile as Avita Cream.

“Comprises/comprising” and grammatical variations thereof when used in this specification are to be taken to specify the presence of stated features, integers, steps or components or groups thereof, but do not preclude the presence or addition of one or more other features, integers, steps, components or groups thereof.

Although specific embodiments of the invention have been described above, it will be clear to the skilled reader that the invention is not restricted to these particular embodiments and the variations and modifications of the invention as particularly described may be made without departing from the scope of the present invention.

1. A substantially homogenous liquid composition capable of percutaneous and/or perungual delivery of one or more physiologically active agents, the composition comprising a volatile solvent, at least one physiologically active agent, and a rate modulating carrier, comprising at least two polymers, one of which is water soluble, and one of which is selected to enable modulation of the rate of delivery of said active agent.

2. The substantially homogenous liquid composition as claimed in claim 1, wherein said rate modulating carrier comprises:
i) a hydrophilic polymer selected from the group consisting of polyvinyl alcohol, hydroxypropylmethyl cellulose, hydroxypropyl cellulose, polyvinyl pyrollidone, carbomer, PVM/MA decadiene cross polymer and hydroxypropylguar and copolymers thereof, said hydrophilic polymer being present in an amount of from 0.001 to 50% w/w of the total liquid composition; and
ii) a hydrophobic polymer being ethyl cellulose and being present in an amount of from 0.00to 50% w/w of the total liquid composition,
the combination of hydrophilic and hydrophobic polymers being selected to enable modulation of the rate of delivery of said physiologically active ingredient.

3. The liquid composition as claimed in claim 1 wherein said physiologically active agent is selected from the group consisting of an anaesthetic, an anti-infective agent or an anti-acne agent.

4. The liquid composition as claimed in claim 3 wherein said physiologically active agent is an anti-acne agent.

5. The liquid composition as claimed in claim 4 wherein said anti-acne agent comprises one or more retinoids.

6. The liquid composition as claimed in claim 5 wherein said retinoid is tretinoin.

7. The liquid composition as claimed in claim 3 wherein said physiologically active agent is an anti-onychomycoses agent.

8. The liquid composition as claimed in claim 7 wherein said anti-onychomycoses agent is an allylamine.

9. A liquid composition as claimed in claim 1 comprising a thickening agent.

10. The liquid composition as claimed in claim 9 wherein said thickening agent is said hydrophilic polymer.

11. The liquid composition as claimed in claim 1 wherein the ratio of said hydrophobic polymer to said active is 1-10,000:10,000-1 on a mass basis.

12. The liquid composition as claimed in claim 11 wherein the ratio of said hydrophobic polymer to said hydrophilic polymer is 1-100:100-1 on a mass basis.

13. The liquid composition as claimed in claim 12 wherein said ratio is 1-10:10-1 on a mass basis.

14. The liquid composition as claimed in claim 1 further comprising one or more skin absorption/penetration enhancers which enhance the absorption and/or penetration of the active agent.

15. The liquid composition as claimed in claim 14 wherein said absorption/penetration enhancer is present in an amount of about 0.1 to 40% w/w of the composition.

16. The liquid composition as claimed in claim 1 where the composition is in the form of a solution.

17. The liquid composition as claimed in claim 1 wherein the composition is in the form of a dispersion comprising a dispersed phase and a continuous phase.

18. The liquid composition as claimed in claim 1 wherein said composition is in the form of a gel.

19. The liquid composition as claimed in claim 17 wherein said dispersed phase comprises any of the group consisting of nanoparticles, microparticles, microcapsules, microspheres, microsponges, or liposomes, which contain the active agent.

20. The liquid composition as claimed in claim 17 wherein said dispersed phase comprises any of the group consisting of nanoparticles, microparticles, microcapsules, microspheres, microsponges, or liposomes, which are coated with the active agent.

21. The liquid composition as claimed in claim 17 wherein said continuous phase includes a hydrophilic polymer.

22. The liquid composition as claimed in claim 17 wherein said continuous phase includes a hydrophobic polymer.

23. The liquid composition as claimed in claim 1 wherein said active agent is dispersed in the composition.

24. The liquid composition as claimed in claim 1 wherein said active agent is dissolved in the composition.

25. The liquid composition as claimed in claim 1 wherein said solvent is present in at least about 50% w/w.

26. The liquid composition as claimed in claim 1 wherein said volatile solvent is selected from the group consisting of ethanol, isopropanol, acetone, water or mixtures thereof.

27. A liquid composition as claimed in claim 1 wherein the total polymer content of the composition is up to about 50% w/w.

28. The liquid composition as claimed in claim 1 wherein said hydrophilic polymer is present in an amount of about 0.05 to 30% w/w.

29. The liquid composition as claimed in claim 28 wherein said hydrophilic polymer is present in an amount of about 0.05 to 10% w/w.

30. The liquid composition as claimed in claim 29 wherein said hydrophilic polymer is present in an amount of about 0.1 to 5.0% w/w.

31. The liquid composition as claimed in claim 1 wherein said hydrophobic polymer is present in an amount of up to about 50% w/w.

32. The liquid composition as claimed in claim 30 wherein said hydrophobic polymer is present in an amount of about 0.001 to 30% of the composition.

33. The liquid composition as claimed in claim 32 wherein said hydrophobic polymer is present in an amount of about 0.05 to 10% w/w.

34. The liquid composition as claimed in claim 33 wherein said hydrophobic polymer is present in an amount of about 0.05 to 6.0%.

35. The liquid composition as claimed in claim 1 wherein upon application to the skin or nail of a subject to the hydrophilic polymer forms a continuous phase, and the hydrophobic polymer is dispersed or soluble therein.

36. The liquid composition as claimed in claim 1 wherein upon application to the skin or nail of a subject the hydrophobic polymer forms a continuous phase, and the hydrophilic polymer is dispersed or soluble therein.

37. The liquid composition as claimed in claim 35 wherein upon application to the skin or nail of a subject said physiologically active agent is contained in said continuous phase.

38. The liquid composition as claimed in claim 35 which is in the form of a dispersion and wherein upon application to the skin or nail of a subject said physiologically active agent is contained in said dispersed phase.

39. The liquid composition as claimed in claim 36 wherein upon application to the skin or nail of a subject said physiologically active agent is contained in said continuous phase.

40. The liquid composition as claimed in claim 36 which is in the form of a dispersion and wherein upon application to the skin or nail of a subject said physiologically active agent is contained in said dispersed phase.

41. A method for the prophylactic or therapeutic treatment of a patient comprising percutaneously or perungually delivering an effective amount of an active agent by application of a composition as claimed in claim 1 to the skin or nail of a subject.

42. The method as claimed in claim 41 wherein said subject is human or animal.

43. The method as claimed in claim 41 wherein said subject is suffering from acne.

44. The method as claimed in claim 41 wherein said subject is suffering from onychomycoses.

45. The method according to claim 41 wherein said composition has anti fungal, anti bacterial, anti viral, anti acne or keratolytic activity.

46. The method as claimed in claim 41 wherein the rate of delivery of said physiologically active agent is adjusted by varying the ratio of said hydrophobic polymer with respect to said active agent.

47. The method as claimed in claim 41 wherein the rate of delivery of said physiologically active agent is adjusted by varying the ratio of said hydrophobic polymer with respect to said hydrophilic polymer.
